(19) United States
(12) Patent Application Publication
(10) Pub. No.: US 2023/0377753 A1
(43) Pub. Date: Nov. 23, 2023

(54) EQUIPMENT AND METHOD TO EVALUATE AND MEASURE BALANCE, FATIGUE AND SLEEPINESS

(71) Applicant: Marco Tulio DE MELLO, Belo Horizonte (BR)

(72) Inventor: Marco Tulio DE MELLO, Belo Horizonte (BR)

(21) Appl. No.: 18/031,026

(22) PCT Filed: Sep. 18, 2021
(86) PCT No.: PCT/BR2021/050399

§ 371 Date: Apr. 10, 2023

(51) Int. Cl. G16H 50/30; G16H 40/67; G16H 10/20; A61B 5/00

(52) U.S. Cl. CPC G16H 50/30; G16H 40/67; G16H 10/20; A61B 5/4806; A61B 5/4023; A61B 2503/20

(57) ABSTRACT

The present patent of invention belongs to the research sector in applied mass spectrometry, and refers, more specifically, to an equipment and method that have the purpose of evaluating and measuring the balance of an individual and determining the index of fatigue and sleepiness, and to send and store the information through the WEB, internet app or on the computer itself. The equipment consists of a box (1) equipped with an electronic display (2), on/off button (3), input (4) for USB connection, attached to a base plate (5) and elastic straps (6), and it is operated via an app, with Bluetooth communication.

### TECHNOLOGICAL SECTOR OF THE INVENTION

In a general way, this patent of invention pertains to the research sector in applied mass spectrometry, and more specifically, it refers to an equipment and method aimed at evaluating and measuring the balance of an individual and determining the fatigue and drowsiness index and forwarding and storing information in WEB, internet applications or in the computer itself.

### PRIOR ART

The sleeping restriction is a commonly observed condition in shift workers and it is highly related to occupational accidents. Van Dongen et al. (2003) noticed that the cognitive performance gets worse and builds up with a chronic sleeping restriction, that is, the less sleeping time, higher is the number of errors and attention lapses.

Dawson e Reid (1997), noticed that individuals awaken for more than 17 hours showed cognitive damage similar to a person who had an alcoholic drink. That is, 19 hours without sleeping, is similar to drinking 6 glasses of beer and 3 glasses of wine or 24 hours without sleeping is equal to 6 glasses of beer and 3 glasses of wine for a man weighing 90 kg.

The sleepiness causes mainly in drivers and workers who work on night shifts and shifts, originates in mental and physical fatigue, lack or restriction of sleeping, due to many hours awaken, starting to have monotony situations, body homeostasis due to reduction of the central temperature, associated to other factors.

Through studies about sleeping and circadian rhythms, it was determined that the conditions in an individual is more subject to sleep, or become fatigued, so that such information are handled and informed through a data system, to avoid that the individual, driver or a worker who works on night shift and shifts, lose the attention during working hours.

In a study by the researchers of this invention “Robust evaluation of time since awakening using force platform posturography” (2014), it was concluded that long periods of wakefulness increase the postural sway. That evidences the restriction of sleeping has a negative influence on physical and cognitive abilities.

There are several scientific articles correlating positively the cognitive deficit with the postural balance, that is, psychomotor impairments are sensed by simple and easily applied tools. For example, Morad et. al (2007) suggests the fatigue caused by lack of sleeping can be evaluated objectively by means of a quick and non-invasive postural test. These authors have concluded that the postural balance can be used as an efficient trial tool for detecting time awaken and the fatigue. In the same reasoning, in another study by the researchers of the invention “Effects of Shift Work on the Postural and Psychomotor Performance of Night Workers” (2016), it was shown that night work per se impairs the postural balance and reduces the performance in surveillance tasks, concentration and attention, demonstrating that simple tasks, such as standing for 30 seconds, are capable of inferring the workers' performance.

Other authors have already validated positively the results of balance tests using a strength platform with a fixed system on the waist, and have concluded that the results are similar and highly reliable and reproductive (Mayagoitia et. al, 2002; Johnson e Trivedi, 2011).

Some inventions disclosed by patent documents have provided possible solutions for the technical problems formerly set out, such as number BR 102013012519-9 published on 21 May 2013, that discloses a process for managing sleeping on the seat and cabin of a vehicle with means of alert, with the object of providing means for collecting and handling data for monitoring his sleepiness conditions by means of a physical platform.

The patent document number BR 112015032507-6 published on 26 Jun. 2014 describes a method for deriving fatigue score, indicative of a sleepiness state, that measures a plurality of signs and derives a score in a weighted and customized way.

And the document number BR 112016011763-8 published on 27 Nov. 2014 consists of a sleeping monitoring device, system and method to monitor the sleeping state of a human being, set up to receive moving data when resting, and determine data of heart rate, breathing data as well as physical activity.

Even then, such solutions do not solve the problems of the individuals herein described. Therefore, it becomes evident that there is a correlation between the postural sway and the psychomotor performance, scientifically justifying the viability of methods that evaluate the postural balance to predict fatigue and avoid errors of attention and occupational accidents. It is expected that with a coherent and suitable fatigue management, workers may have greater willingness, less risk of accident and use their potentialities to maximize their productivity and performance at work and are not tied to orthodox systems that do not dynamize and potentialize their mental and physical abilities.

### Novelties and Purposes of the Invention

The equipment is connected to a mobile phone (via app) and it was developed to evaluate the balance, physical fatigue and sleepiness of a human being, aimed at forwarding the collected data to the cloud, which carries out the calculations of fatigue and sleepiness, and returns the obtained data to the user or client, via app or Web portal, accessing on his own computer.

The equipment shows advantages such as calculating the fatigue level of the worker through predictive, proactive actions, and constant management of the indicators; forwarding the results via SMS to the worker informing him about his current level of fatigue; making available tips by means of messages of how to minimize the fatigue; monitoring the fatigue level of the driver for “online” follow-up via site, e-mail or SMS of the manager/supervisor and can be integrated to other physical platforms.

### DESCRIPTION OF THE ATTACHED DRAWINGS

So that this invention is fully understood and practiced by any technician in this technological sector, it will be described in a clear, concise, and sufficient way, based on the attached drawings, which illustrate and subsidize listed below:

FIG. 1 shows a front view of the equipment;

FIG. 2 shows a side view of the equipment;

FIG. 3 shows a front and back view of the vest adjustable with the equipment;

FIG. 4 shows a front and back view of the elastic band adjustable with the equipment;

FIG. 5 shows the graph issued in the Web portal of the fatigue information along the time of the workers.

FIG. 6 shows the graph with information about the workers; more specifically regarding the projection of the fatigue index over time;

FIG. 7 shows the procedure scheme for evaluating the individuals.

### DETAILED DESCRIPTION OF THE INVENTION

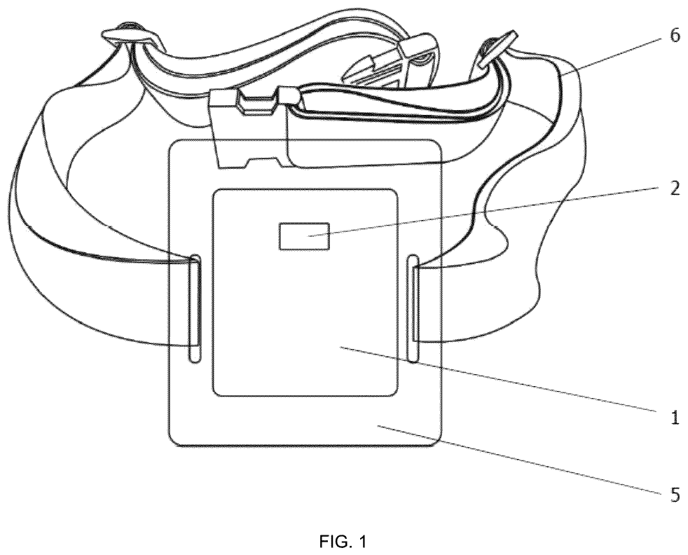

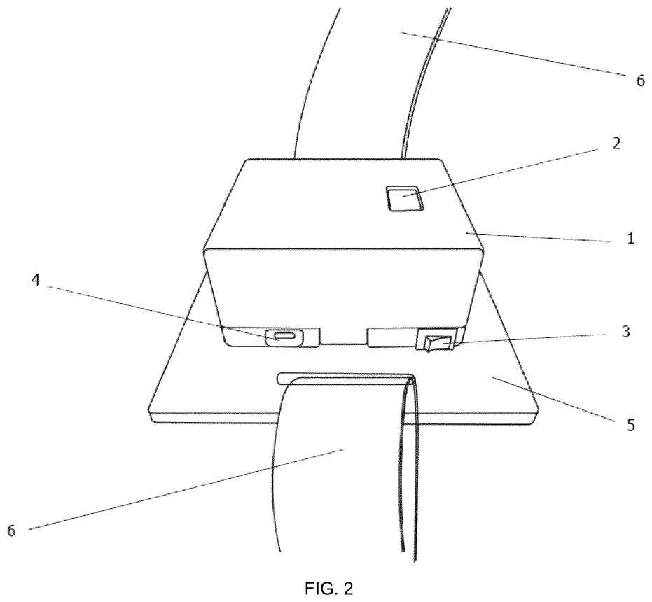

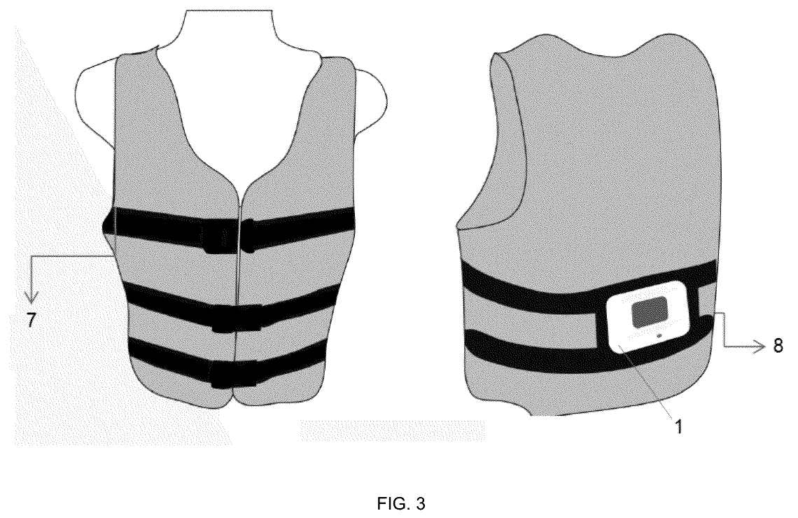

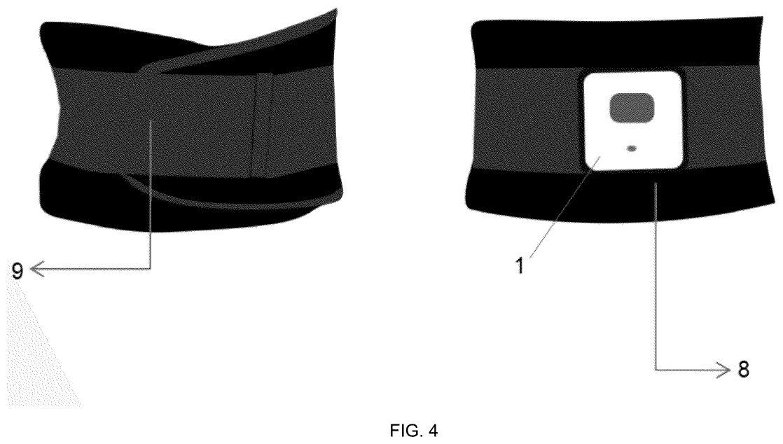

According to FIGS. 1 and 2, the equipment to evaluate and measure the balance, fatigue and sleepiness, object of this invention, is a device to be worn on the waist, more specifically on the lumbar spine for basal measurement, which consists of a polymeric material box (1) provided with an electronic visor (2), on/off button (3), input (4) for USB connection, fixed to a base plate (5) and elastic straps (6), that is operated by means of an app, with Bluetooth communication. Said button (3) is based on the operation of a switch, with light touch precision and without hardness and said input (4) for loading is projected to be a fixed input and with a firmer fitting, obtaining more protection for the connection input for loading, reducing the risk of having parts disengage. According to FIGS. 3 and 4, the equipment can be applied next to the waist through a vest adjustable with plastic hooks (7) and a placing pocket (8) or through an elastic band with press-stud adjustable to the body shape (9) and pocket for placing the apparatus (8).

The information safety is guaranteed by the server, which stores the processed data from the raw data generated by the equipment, which are protected by the access control implemented on higher levels of the system, where the safety on the hardware level should be guaranteed by the individual having physical access to the product, and the raw data is stored on a memory card embedded in the hardware, and is also stored in the server when forwarded for processing.

There are several ways for evaluating and observing the human behaviour regarding the aspect fatigue. It was decided to develop and improve assessments that address the biological and behavioral profile of workers, through questionnaires; the genetic profile evaluation, regarding the genetic characteristic of the worker, and the fatigue evaluation, using app, Web portal and the current equipment described. The instruments related to questionnaires are largely validated in the literature, through several scientific publications. However, it seeks to carry out the automatic correction of such questionnaires, thus, facilitating its application, correction, processing time and number of people involved in this evaluation, contributing with the cost for the company relating to human resources. The development of applications and portal web brings easiness, convenience and speed in the evaluations which it proposes to carry out.

Such periodic measurements and evaluations, aim at allowing or not the individual to begin his working hours, and that during such period, through the data available, he acts safely mainly in his routine, as for example driving people or cargos in the case of drivers.

### Example of Application of the Equipment

The equipment can be deployed for workers who drive cargo automotive vehicles, aiming at improving the management of health and occupational safety.

It provides to the contracting company, on load, the equipment, mobile phone, data transmission chip and licenses for using the software. The system for warning and readiness to work is applicable to all the employees performing critical/special activities, notably driving light and heavy automotive vehicles, and to all other workers acting at critical areas and working on night shifts and in shifts.

A Web managing system is used, including access permissions, validated by the Information Technology (IT) teams. Such system has the aim of predicting the real fatigue, when arriving for the working hours and carries out an algorithm calculation, already validated, to predict the fatigue until the end of the working hours.

The fatigue monitoring consists of the interface of two equipment the application or portal Web and the equipment.

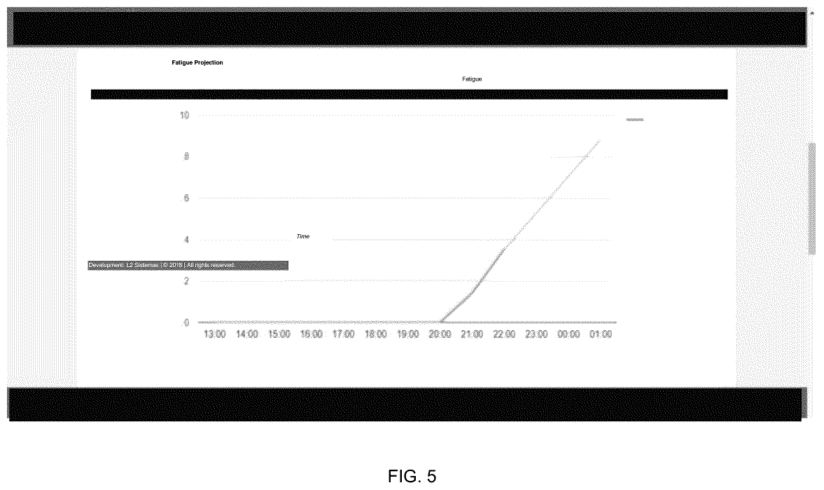

At the Web portal the managers will have access to information about the workers (work schedule, level of current fatigue, history), report issue, alteration and inclusion of registrations, as shown in FIG. 5.

The main functionalities of the portal Web are:
Controlling access to the system in authentication and authorization of the user;
Registration of customer data, as well as their respective parameterizations;
Registering data of the users/workers, as well as their respective characteristics;
Filling in questionnaire when starting working hours;
Fatigue projection graphs;
warnings/alerts;
Control panel.

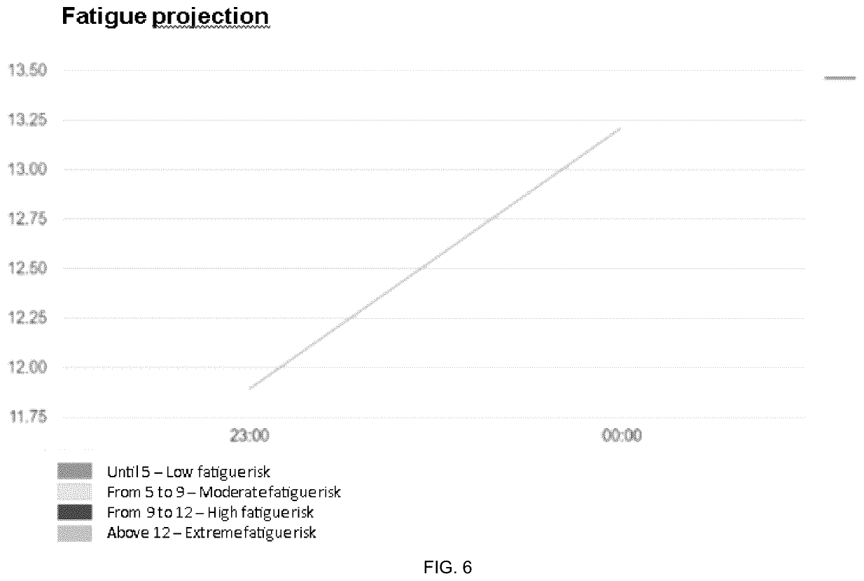

The app for the mobile phone has the function of analysing and projecting the fatigue level of the worker during the working hours. The workers registers filling in personal information such as, for example, age, marital status and sleeping profile. For fatigue evaluation, the user enters information about the working hours, carries out the evaluation in the equipment and the application shows the current fatigue score and projections of fatigue risk (scores) by means of graphs, making evident the critical moment for performing laboral activities, according to FIG. 6. Such system, if it is the option of the client to use, should be installed in the mobile phones of the collaborators. Such system and evaluation provides a score, which is classified as:
Normal fatigue (score up to 5);
Moderate fatigue (score between: 5.09 and 9.0);
High fatigue (score between 9.01 and 12.0);
Extreme fatigue (score higher than 12.01).

The company/client will determine the maximum acceptable point for the fatigue of his collaborators, and thus, during the registration of the company in the system it will enter the maximum allowed score.

During the working period, specifications and notifications online of the level of predicted fatigue are provided, for each collaborator, and other information can also be implemented, at the discretion of the company, such as:

Per collaborator:
Fatigue average score when starting the working hours;*
Fatigue average score at the end of the working hours;*
Fatigue average score during the working hours;*
Estimated total time agreed upon before the working hours;*
Estimated total time agreed upon at the end of the working hours;*
Among other implementations which can be analysed, in a budget to be later defined and not shown on the commercial proposal which will be forwarded together with this technical proposal.

Global analysis of all the collaborators:
Fatigue average score when starting the working hours;*
Fatigue average score at the end of the working hours;*
Fatigue average score during the working hours;*
Estimated total time agreed upon before the working hours;*
Estimated total time agreed upon at the end of the working hours;*
Refers to all previous working days, where the evaluation with the equipment and application was carried out.

The input of data regarding the registration of the users can be carried out by the Mobile app or in the Web portal. The registration covers data referring to the general aspect of the works, health, family and work.

The equipment is designed to be used on the waist in the lumbar region (FIGS. 3 and 4). It consists of a box (1) fixed on a base plate (5) and elastic straps (6) and will be supplied to each workplace.

The equipment should be used only during the fatigue evaluation; by means of balance, it is capable of inferring the fatigue level. A more fatigued individual increases even more the body sway. Five test evaluations shall be carried out during different moments of the working hours for the algorithm to identify and determine the individual sway pattern in order to infer the fatigue level at the evaluation moment. The equipment is placed around the waist of the individual evaluated for collecting the fatigue level and readiness for the occupational activities.

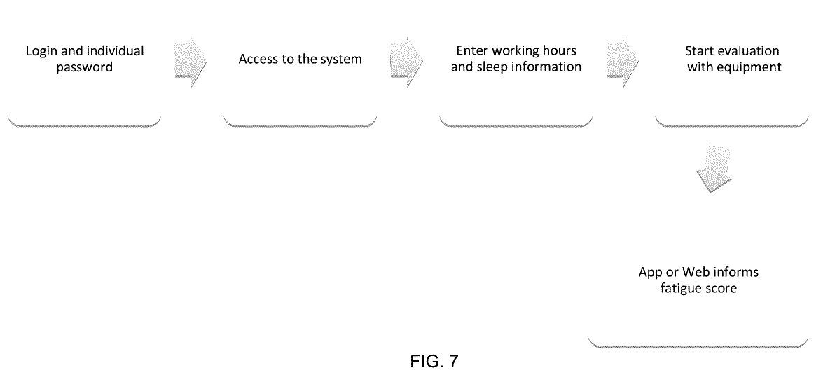

When the workers arrive at the job site to start the working hours they should carry out the evaluation in the equipment. According to FIG. 7, to access the app, the collaborators should use the login and individual password, previously registered. After carrying out the registration the evaluated individual (collaborator/employee) shall carry out, before 3 periods of working hours, 3 to 5 basal evaluations at different working days, with a minimum interval of 20 h between each evaluation and after having rested enough for his recovery (between 7 h and 9 h of sleep), preferably night sleep. Only after the basal evaluations is that the data collection will be released for evaluating the fatigue of the worker. Since the fatigue score is individual, the artificial intelligence shall have, as reference, the data collected in the basal evaluations. It is important the basal collections happen within two hours after the collaborator has woken up, so that the pattern and the reference will be considered with the worker rested. If they are collected at a higher term, the fatigue scores will be mitigated, as there will be a comparison with a fatigue level higher than the basal level.

The registered collaborator shall fill in data regarding his sleep and working hours and give the command to start the fatigue evaluation in the equipment. The evaluation lasts 60 seconds (divided in two collections of 30 seconds, simultaneous), the collaborator should remain quiet in a bipodal position during the evaluation period. At the end on the smartphone screen, or on Web, the fatigue score will be shown.

Though the equipment evaluates quantitatively the level of attention of the worker, on its turn it allows to infer the state of alert and vigilance to perform the laboral activities by means of postural balance. Besides, it is a system able to predict the fatigue level of the collaborator in real time and by means of score it is possible to classify the worker as apt or not to start the working hours safely. However, the actions to be performed after supplying the fatigue score, to the client, are his responsibility. To this end, it must have an action policy to assist the collaborators who show an inadequate score.

Through the portal, managers can access the evaluations of all evaluated workers. The system allows monitoring the fatigue level of the workers who are working, as well as the access of histories of the fatigue projections. Moreover, in the portal it is possible to add or remove users, add or remove working hours/shifts, issue individual and/or group reports and register shifts and working risk profiles.

Through the portal, the managers may have exclusive access to information about his team as, for example, registered users, working hours and fatigue scores. This favors feedback, information management as well as assertive decisions aimed at better results and safer operations.

In the Web portal, the managers are able to issue general monthly reports (PDF or Excel format), the relation of evaluated workers, amount of evaluations performed and fatigue projections.

The safety of the information is guaranteed by the server which stores the processed data as from the raw data generated by the equipment. Such data are protected by access control implemented at the higher levels of the system. The safety at the hardware level should be guaranteed by the individual who has physical access to the product. And the raw data are stored on a memory card embedded in the hardware, as already mentioned. Besides, it is also stored in the server when forwarded for processing.

It is important to emphasise that the figures and description carried out are not intended to limit the forms of carrying out the inventive concept herein proposed, but rather to illustrate and make understandable the conceptual innovations revealed in this solution. Thus, the descriptions and images should be interpreted in an illustrative and not limiting way, other equivalent or analogue forms can exist for implementing the inventive concept herein disclosed that do not deviate from the spectrum of protection outlined in the proposed solution.

1- EQUIPMENT TO EVALUATE AND MEASURE BALANCE, FATIGUE AND SLEEPINESS, characterized by being composed of box (1) with electronic display (2), on/off button (3), port for USB connection (4), base plate (5) and elastic straps (6), and calculate the level of fatigue by proactive, predictive actions and constant management of indicators.

2- EQUIPMENT TO EVALUATE AND MEASURE BALANCE, FATIGUE AND SLEEPINESS, according to claim 1, and characterized by been applied around the waist by means of a vest adjustable by plastic hooks (7) and a pocket for placement (8) or by an elastic strap with pressure button adjustable to the shape of the body (9) and pocket for placing the device (8).

3- METHOD TO EVALUATE AND MEASURE BALANCE, FATIGUE AND SLEEPINESS, characterized by the fact that for accessing the application, having to log in with an individual password and, after completing the registration, the evaluated person performs, prior to 3 periods of the working day, 3 to 5 baseline assessments on different working days, with a minimum interval of 20 hours between each evaluation and after having rested; for the registered employee to fill in data about their sleep and workday and give the command to start the assessment of fatigue on the equipment and the assessment lasts 60 seconds, divided into two simultaneous 30 second collections, being in a bipedal position during the period evaluation and at the end the fatigue score will be displayed on the smartphone screen or on the Web.

4- METHOD TO EVALUATE AND MEASURE BALANCE, FATIGUE AND SLEEPINESS, according to claim 3, characterized by baseline collections occur within a period of up to 2 hours after the employee has woken up.

5- METHOD TO EVALUATE AND MEASURE BALANCE, FATIGUE AND SLEEPINESS, according to claim 1, and characterized by sending results via SMS to the evaluated person informing his current level of fatigue and providing tips through messages on how to minimize fatigue.

6- METHOD TO EVALUATE AND MEASURE BALANCE, FATIGUE AND SLEEPINESS, according to claim 1, and characterized by online monitoring of the fatigue level of the subject via website/via email or SMS from the manager and can be integrated with other physical platforms.
